(19) United States
(12) Patent Application Publication
(10) Pub. No.: US 2002/0193379 A1
(43) Pub. Date: Dec. 19, 2002

(54) Compositions and methods for decreasing the risk of or preventing neural tube disorders in mammals

(75) Inventors: Andrew J. Copp, London (GB); Geoffrey Allan, Richmond, VA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Insmed Incorporated

(21) Appl. No.: 10/144,767

(22) Filed: May 15, 2002

Related U.S. Application Data
(60) Provisional application No. 60/295,598, filed on Jun. 5, 2001.

(51) Int. Cl. A61K 31/525; A61K 31/045

(52) U.S. Cl. 514/251; 514/729

(57) ABSTRACT

This invention relates to compositions and methods for decreasing the risk of or preventing neural tube disorders in mammals. These compositions contain an effective amount of D-chiro-inositol or a combination of effective amounts of D-chiro-inositol and folic acid.

This application claims priority to U.S. Provisional Patent Application Serial No. 60/295,598, filed Jun. 5, 2001, which application is hereby incorporated by reference in its entirety.

### BACKGROUND OF THE INVENTION

1. Field of the Invention

2. Related Art

This invention relates to compositions and methods for decreasing the risk of or preventing neural tube disorders characterized by spina bifida, spina bifida anterior, spina bifida aperta, spina bifida cystica, spina bifida occulta, spina bifida posterior, anencephaly, exencephaly, meningomyelocele and/or encephalocele in mammals.

Neural tube defects (NTDs) are severe malformations of the central nervous system and axial skeleton resulting from disturbances to the process of neurulation in mammals. Neurulation is the process in embryonic development denoted by the folding of the edges of the embryonic neural plate toward each other and fusing to form the neural tube followed by the closing of the open caudal end of the neural tube, or posterior neuropore.

The neural tube is the progenitor structure of all central nervous system and axial skeletal components, and the failure of the edges of the neural plate or posterior neuropore to completely close and fuse results in the neural tube defects characterized by spina bifida, spina bifida anterior, spina bifida aperta, spina bifida cystica, spina bifida occulta, spina bifida posterior, anencephaly, exencephaly, meningomyelocele and/or encephalocele. NTDs are classified as open or closed defects: open defects, such as spina bifida, anencephaly, and meningomyelocele are uncovered lesions arising from failures of the primary phase of neurulation; closed defects, such as encephalocele, are covered by skin and arise from failures following primary neurulation. A spectrum of other conditions associated with neural tube closure defects, including hypoplasia or aplasia of cranial nerve nuclei, obstruction of the flow of cerebrospinal fluid flow within the ventricular system, cerebellar dysplasia, disordered migration of cortical neurons, fusion of the thalami, genesis of the corpus callosum, and complete or partial agenesis of the olfactory tract and bulb, found in children suggest that such malformations may seriously effect intellectual outcome (Gilbert et al., 1986).

NTDs occur commonly in human development, with a typical prevalence of 0.5-2.0 per 1000 births, and with a higher frequency among spontaneously aborted fetuses (Copp, 1998). While NTDs are associated with genetic abnormalities, particularly trisomies of chromosomes 18 and 13 and X chromosome monosomies in humans, no obvious pattern of Mendelian inheritance has been established (Copp, 1998). Open NTDs in humans result from disturbance of neural tube closure during neurulation between the 17ₜₕ and 30ₜₕ day after ovulation (Coerdt et al., 1997). Up to 70% of NTDs can be prevented by folic acid supplementation in early pregnancy, whereas the remaining 30% of NTDs are resistant to folic acid (Wald et al., 1991).

The curly tail mouse is an animal model for folic acid resistant NTDs (Greene and Copp, 1997). Myo-inositol, a deficiency of which was found to promote NTDs in cultured rat embryos (Cockroft, 1988) and in cultured curly tail mice embryos (Cockroft, 1992), is also capable of significantly reducing the incidence of spinal NTDs both in cultured embryos and in offspring of curly tail mice treated with myo-inositol during pregnancy (Greene and Copp, 1997). Additionally, myo-inositol has been shown to substantially reduce the incidence of NTDs resulting from the teratogenic effects of hyperglycemia; NTDs normally occur in embryos of diabetic mothers at a rate four to five times higher than that observed in the general population (Reece et al., 1997).

However, large doses of myo-inositol are required to prevent NTDs in curly tail mice. This may contraindicate the use of myo-inositol in pregnant mammals since inositol may stimulate uterine contractions (Colodny et al., 1998). The finding that insulin-mediator complexes containing chiro-inositol are 50-100 times more active than myo-inositol complexes in stimulating glucose incorporation into glycogen (Huang et al., 1993) suggested that trace amounts of chiro-inositol may have been the active ingredient in the myo-inositol preparations used in those studies.

### SUMMARY OF THE INVENTION

It has been found that certain isomers of inositol, namely D-chiro-inositol and derivatives and metabolites thereof and compounds containing D-chiro-inositol or a derivative or metabolite thereof, have significant effects on mammalian neurological development. More specifically, D-chiro-inositol and derivatives and metabolites thereof and compounds containing D-chiro-inositol or a derivative or metabolite thereof, when administered to curly tail mice predisposed to NTDs otherwise resistant to folic acid intervention, substantially decreased the incidence of neural tube disorders characterized by spina bifida, spina bifida anterior, spina bifida aperta, spina bifida cystica, spina bifida occulta, spina bifida posterior, anencephaly, exencephaly, meningomyelocele and/or encephalocele.

In conjunction with these results is the finding that D-chiro-inositol and derivatives and metabolites thereof and compounds containing D-chiro-inositol or a derivative or metabolite thereof, when administered to curly tail mice predisposed to NTDs otherwise resistant to folic acid intervention, decreases the risk of or prevents abnormal neurulation characterized by neural tube disorders, enhances posterior neuropore closure, decreases the risk of failure of posterior neuropore closure, decreases the ratio of posterior neuropore length to crown-rump length, decreases the risk of or prevents failure of the neural tube to close at the rostral end in mammals which results in NTDs characterized by spina bifida, and decreases the risk of or prevents failure of the neural tube to close at the caudal end in mammals which results in NTDs characterized by anencephaly.

It is to be understood that both the foregoing general description and the following detailed description are exemplary and explanatory only and are intended to provide further explanation of the invention as claimed.

### BRIEF DESCRIPTION OF THE DRAWINGS

FIG. 1 is a table showing the 5.6-fold decrease in mean posterior neuropore to crown-rump length in embryonic curly tail mice following 24 hours of treatment with D-chiro-inositol in vitro.

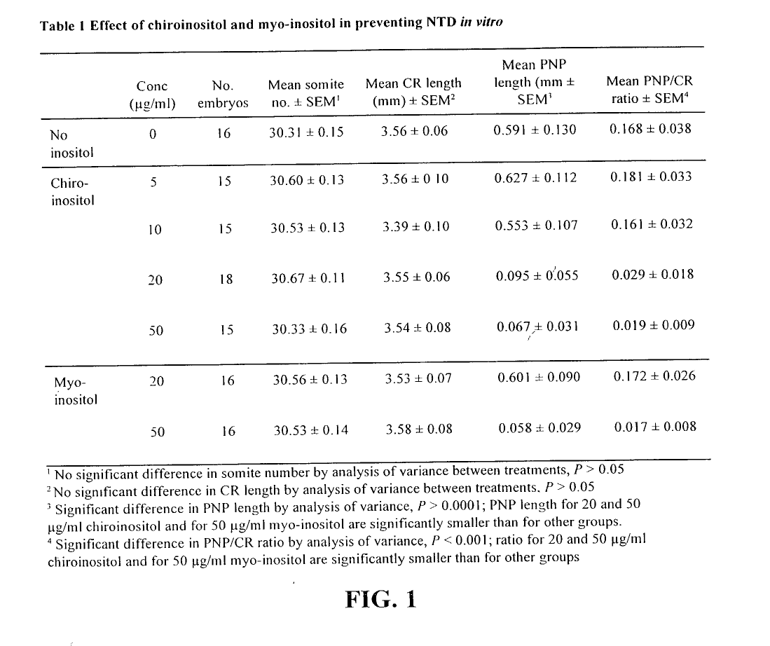

FIGS. 2A and 2B are tables showing the 9-fold decrease in the number of curly tail mouse embryos showing NTDs characterized by spina bifida following four to ten days of treatment with D-chiro-inositol by subcutaneous administration.

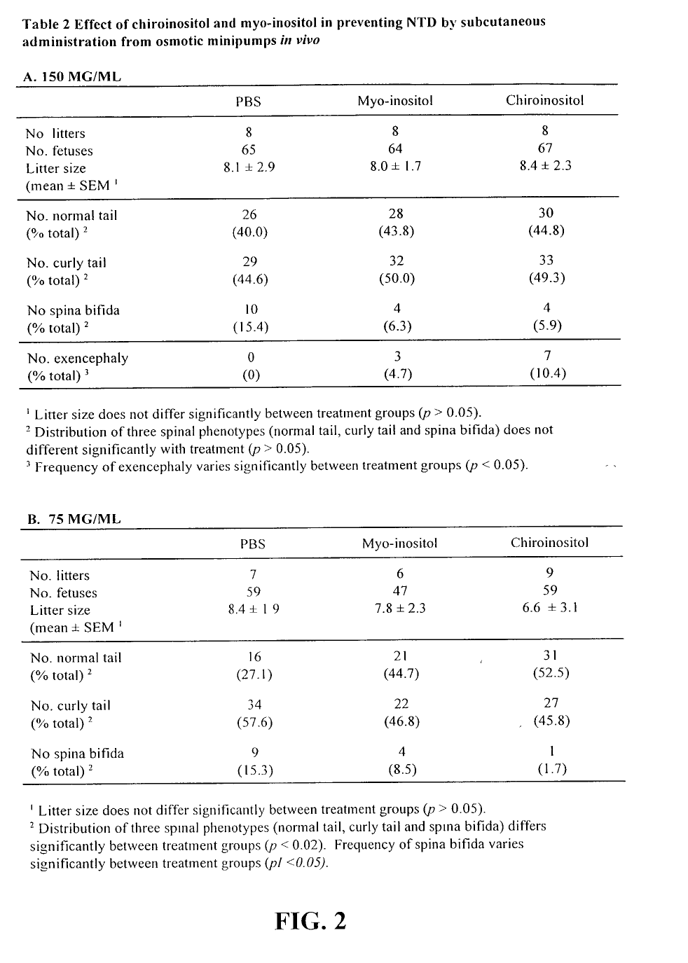

FIG. 3 is a table showing the 8-fold decrease in the number of curly tail mouse embryos showing NTDs characterized by spina bifida following three days of treatment with D-chiro-inositol by oral administration.

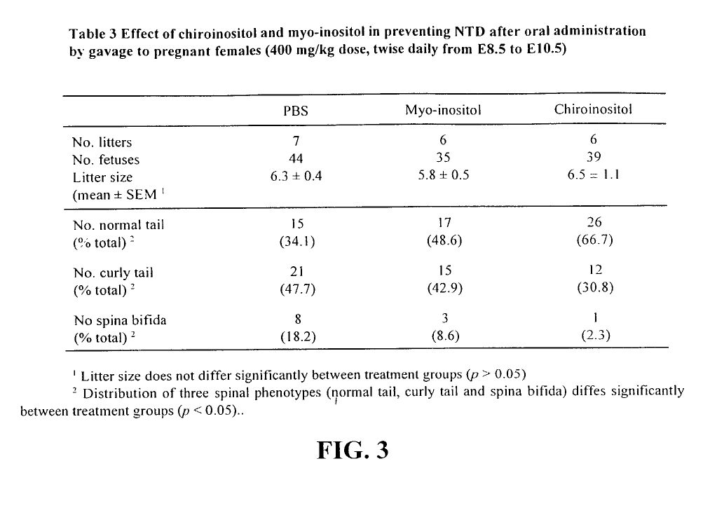

FIG. 4 is a graph showing the 6-fold decrease in mean posterior neuropore length in embryonic curly tail mice following 24 hours of treatment with either myo-inositol or D-chiro-inositol in vitro.

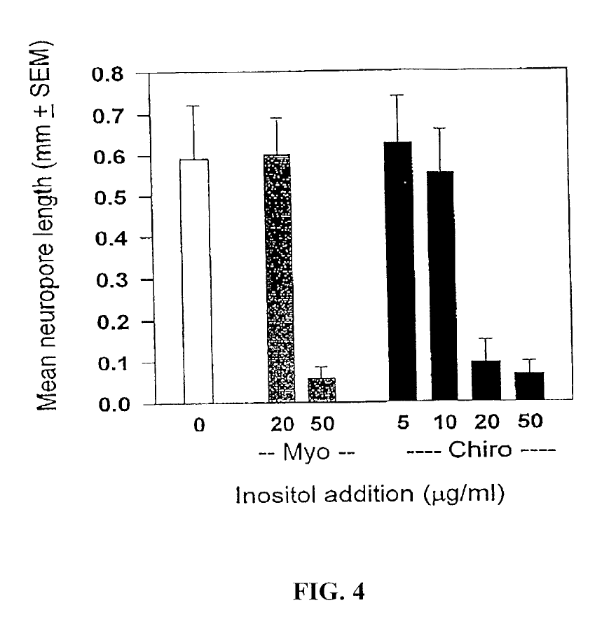

FIGS. 5A and 5B are graphs showing the 9-fold decrease when compared to control and the 4-fold decrease when compared to myo-inositol in the number of curly tail mouse embryos showing NTDs characterized by spina bifida following four to ten days of treatment with D-chiro-inositol by subcutaneous administration.

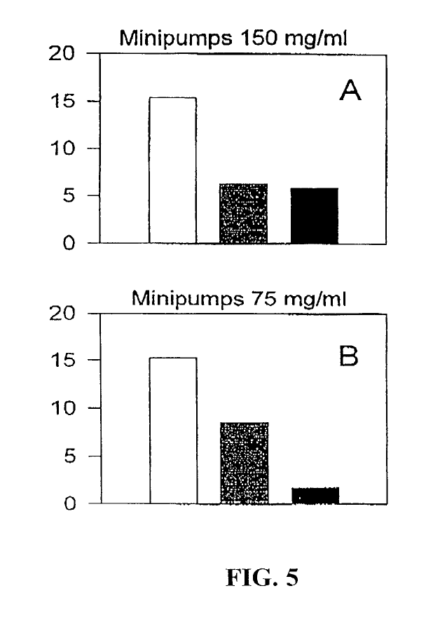

FIG. 6 is a graph showing the 8-fold decrease when compared to control and the 3-fold decrease when compared to myo-inositol in the number of curly tail mouse embryos showing NTDs characterized by spina bifida following three days of treatment with D-chiro-inositol by oral administration.

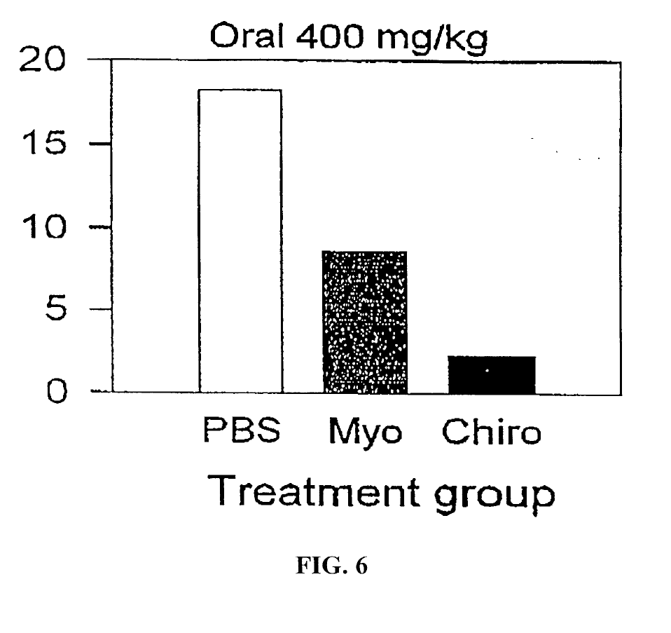

FIGS. 7A and 7B are graphs showing (A) Exposure of curly tail embryos in culture to myo-inositol or D-chiro-inositol causes a dose-dependent reduction in posterior neuropore length. Embryos treated with PBS (control) are indicated as “0.” (B) Administration of myo- or D-chiro-inositol to pregnant curly tail females by slow release from sub-cutaneously implanted minipumps reduces the frequency of spina bifida at doses of 72 and 144 μg/g body weight/day, but not at 29 μg/g body weight/day. A similar preventive effect of inositol is seen with oral dosing at 800 μg/g body weight/day.

### DETAILED DESCRIPTION OF THE INVENTION

In a first preferred embodiment the present invention is directed to compositions and methods for decreasing the risk of or preventing neural tube disorders characterized by spina bifida, spina bifida anterior, spina bifida aperta, spina bifida cystica, spina bifida occulta, spina bifida posterior, anencephaly, exencephaly, meningomyclocele and/or encephalocele in a mammal, which comprises an effective amount of D-chiro-inositol, or a suitable derivative or metabolite thereof, or a compound containing D-chiro-inositol or a derivative or metabolite thereof, and an acceptable carrier.

The inventive composition comprises an effective amount of D-chiro-inositol, or a suitable derivative or metabolite thereof, or a compound containing D-chiro-inositol or a derivative or metabolite thereof, and an acceptable carrier. Preferably, the inventive composition comprises an effective amount of D-chiro-inositol or a compound containing D-chiro-inositol.

The inventive method comprises administering to a mammal an effective amount of D-chiro-inositol, or a suitable derivative or metabolite thereof or a compound containing D-chiro-inositol or a derivative or metabolite thereof.

While the inventive composition preferably comprises D-chiro-inositol or a compound containing D-chiro-inositol, suitable derivatives and/or metabolites of D-chiro-inositol, or compounds containing derivatives or metabolites of D-chiro-inositol, may also be employed.

As used herein, a “suitable derivative or metabolite” of D-chiro-inositol is a compound based on or derived from the D-chiro-inositol moiety.

Illustrative examples of suitable derivatives and metabolites of D-chiro-inositol include, but are not limited to, the following: D-chiro-inositol phosphates; D-chiro-inositol esters, preferably acetates; D-chiro-inositol ethers, preferably lower alkyl ethers; D-chiro-inositol acetals; and D-chiro-inositol ketals.

As used herein, a “compound containing D-chiro-inositol” is any compound that contains the D-chiro-inositol moiety. Illustrative examples of D-chiro-inositol containing compounds include, but are not limited to, the following: polysaccharides containing D-chiro-inositol and one or more additional sugars, such as glucose, galactose and mannose, or derivatives thereof, such as glucosamine, galactosamine and mannitol; D-chiro-inositol phospholipids; and complexes or chelates of D-chiro-inositol with one or more metal ions and the like. Specific examples include, but are not limited to, genuine D-chiro-inositol, pinitol (a methyl ether of D-chiro-inositol), fagopyritols, amino disaccharides as described in U.S. Pat. No. 5,652,221.

The active agent in the inventive composition (i.e. D-chiro-inositol or a suitable derivative or metabolite thereof or a compound containing D-chiro-inositol, or a derivative or metabolite thereof) may be used alone or in admixture with one or more additional active agents. For example, a composition according to the first embodiment of the present invention may include D-chiro-inositol and a compound containing D-chiro-inositol in admixture.

As used herein, an “acceptable carrier” is a non-toxic solid, semisolid or liquid filler, diluent, encapsulating material or formulation auxiliary of any type known to those skilled in the art for use in pharmaceuticals.

When administered to a mammal, the inventive compositions may be administered orally, rectally, parenterally, intrasystemically, intravaginally, intraperitoneally, topically (as by powders, ointments, drops or transdermal patch), bucally, or as an oral or nasal spray. Preferably, the inventive compositions are administered orally, for example in the form of a tablet or capsule.

As used herein, the term “parenteral” refers to modes of administration which include intravenous, intramuscular, intraperitoneal, intrasternal, subcutaneous and intraarticular injection and infusion.

The compositions of the present invention are also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al.) or poly-D-(-)-3-hydroxybutyric acid (EP 133,988).

Sustained-release compositions also include liposomally entrapped compounds. Liposomes containing one or more of the compounds of the present invention may be prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Å) unilamellar type in which the lipid content is greater than about 30 mol. percent cholesterol, the selected proportion being adjusted for the optimal therapy.

For parenteral administration, in one embodiment, the composition of the present invention is formulated generally by mixing an effective amount of the active agent at the desired degree of purity, in a unit dosage injectable form (solution, suspension, or emulsion), with an acceptable carrier, i.e., one that is non-toxic to recipients at the dosages and concentrations employed and is compatible with other ingredients of the formulation. For example, the formulation preferably does not include strong oxidizing agents and other compounds that are known to be deleterious to the active agent.

Generally, the formulations are prepared by contacting the active agent uniformly and intimately with liquid carriers or finely divided solid carriers or both. Then, if necessary, the product is shaped into the desired formulation. When the carrier is a parenteral carrier, it is preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG. The compositions of the present invention are typically formulated in such vehicles at a concentration of active agent of about 1 mg/mL to 240 mg/mL, preferably 30 to 120 mg/mL, and even more preferably at 75 mg/mL.

It will be understood that the use of certain of the foregoing excipients, carriers, or stabilizers may result in the formation of salts depending upon the particular substitutent(s) on the active agent.

The compositions of the present invention ordinarily will be stored in unit or multi-dose containers, for example, sealed ampules or vials, as an aqueous solution or as a lyophilized formulation for reconstitution. As an example of a lyophilized formulation, 10-mL vials are filled with 5 mL of sterile-filtered 1% (w/v) aqueous solution, and the resulting mixture is lyophilized. The infusion solution is prepared by reconstituting the lyophilized composition using bacteriostatic Water-for-Injection.

The compositions of the present invention will be formulated and dosed in a fashion consistent with good medical practice, taking into account the clinical condition of the individual patient (especially the side effects of treatment with the active agent), the site of delivery of the composition, the method of administration, the scheduling of administration, and other factors known to practitioners. The “effective amount” of active agent for the purposes of the present invention is determined in view of such considerations. Those skilled in the art can readily determine empirically an appropriate “effective amount” for a particular patient.

The key factor in selecting an appropriate dose is the result obtained, as measured, for example, by decreasing the risk of or preventing neural tube disorders characterized by spina bifida, spina bifida anterior, spina bifida aperta, spina bifida cystica, spina bifida occulta, spina bifida posterior, anencephaly, exencephaly, meningomyelocele and/or encephalocele in the patient. The length of treatment needed to observe changes and the interval following treatment for responses to occur appears to vary depending on the desired effect.

As a general proposition, the total effective amount of D-chiro-inositol containing compound active agent administered per dose will be in the range of about 3 mg/kg/day to about 300 mg/kg/day of mammalian patient body weight, although, as noted above, this will be subject to therapeutic discretion. More preferably, this dose is at least 5 mg/kg/day, and most preferably for humans between about 5 and about 180 mg/kg/day.

In another embodiment, an effective amount of folic acid is included in the composition. Since the occurrence of all neural tube defects is desired and since the corrective agent cannot be predicted a priori, the preferred formulations include both D-chiro-inositol and folic acid as active components either alone or as part of a prenatal vitamin formulation. Folic acid is generally given in an amount between about 0.25 mg/day and about 6.0 mg/day, preferably from about 0.5 mg/day to about 5.0 mg/day, most preferably from about 1.0 mg/day to about 4.0 mg/day. Minimum requirements of folic acid are in the range of 50 μg/day, and increase 3 to 6 times during pregnancy and/or lactation. The U.S. recommended daily allowance for pregnant women is 400 μg/day, and the average pharmacological replacement dose is between 1 and 5 mg/day. Most prenatal vitamins contain 1 mg of folic acid. Thus in a preferred embodiment, a quantity of D-chiro-inositol is formulated in a prenatal vitamin formulation containing 1 mg of folic acid.

The total body store of folic acid is about 5 mg. When a folic acid-deficient patient is treated, reversal of the deficiency begins rapidly (reticulocytosis within 4 days) and resolves within 2 months. If folic acid is administered at a rate of only 50 μg day, assuming no dietary or other intake, signs of folic acid deficiency are manifest after an approximately 3 month lag time. In cases of increased bodily folic acid requirements, such as pregnancy or lactation, this time frame is shortened to 2 to 4 weeks. Fortunately, folic acid supplementation in otherwise healthy young women who have such increased folic acid needs is an accepted practice.

Folic acid has not been reported to cause adverse effects when administered in reasonable, pharmacological doses. The only reported adverse reaction for folic acid is a decreased level of plasma zinc in the case of prolonged high-dose administration.

In a most preferred embodiment, the inventive compositions are formulated for oral delivery according to the methods known to those skilled in the art. For example, the active agent is combined with suitable sweetening agents, flavoring agents, coloring agents and preserving agents, in order to obtain a palatable preparation. Tablets, capsules, powders, granules, and the like for oral administration may contain the active agent in admixture with acceptable additives or excipients. Such forms may be prepared by mixing the active agent(s) with one or more additives and excipients, such as inert diluents, granulating agents, disintegrating agents, binding agents and/or lubricating agents, under suitable conditions.

Acceptable additives and excipients are known to those skilled in the art (see, e.g., Remington's Pharmaceutical Sciences, 18th ed., A. Gennaro, ed., Mack Publishing Company, Easton, Pa. (1990)). Illustrative examples of acceptable additives and excipients for oral compositions include, but are not limited to, water, non-fat dry milk, maltodextrin, sugar, corn syrup, sodium caseinate, soy protein isolate, calcium caseinate, potassium citrate, sodium citrate, tricalcium phosphate, magnesium chloride, sodium chloride, lecithin, potassium chloride, choline chloride, ascorbic acid, potassium hydroxide such as calcium carbonate, sodium carbonate, lactose, calcium phosphate, carrageenan, vitamin E, zinc sulfate, ferrous sulfate, macinamide, biotin, vitamin A, calcium pantothenate, copper gluconate, magnesium sulfate, vitamin K, potassium iodide, vitamin D, vanillin, cocoa, polysorbate 80, polysorbate 60, magnesium oxide, riboflavin, pyridoxine hydrochloride, cyanocobalamin, aspartame, thiamine, cellulose, methyl cellulose, hydroxypropyl methylcellulose, alginate, polyoxyelthylene sorbitol monooleate, polyoxyethylene stearate, gum acacia, gum tagacanth, polyvinylpyrrolidone, gelatin, calcium carbonate, calcium phosphate, kaolin, starch, and the like.

When administered orally, the inventive composition preferably contains from about 1 mg to about 1200 mg of active ingredient. In the case of D-chiro-inositol, the inventive composition preferably contains from about 10 mg to about 1000 mg of DCI, more preferably about 30 mg to about 1000 mg, and most preferably about 100 mg to about 1000 mg. If folic acid is present in the composition, it is generally present in an amount between about 0.25 mg and about 6.0 g, preferably from about 0.5 mg to about 5.0 mg, most preferably about 1.0 mg.

A second preferred embodiment of the present invention is directed to compositions and methods for decreasing the risk of or preventing neural tube disorders caused by diabetes or hyperglycemia characterized by spina bifida, spina bifida anterior, spina bifida aperta, spina bifida cystica, spina bifida occulta, spina bifida posterior, anencephaly, exencephaly, meningomyelocele and/or encephalocele in a diabetic or hyperglycemic mammal.

In this embodiment, the inventive composition comprises an effective amount of D-chiro-inositol, or a suitable derivative or metabolite thereof, or a compound containing D-chiro-inositol or a derivative or metabolite thereof, and an acceptable carrier. Preferably, the inventive composition comprises D-chiro-inositol.

The inventive method comprises administering to a mammal in need thereof an effective amount of D-chiro-inositol, or a suitable derivative or metabolite thereof, or a compound containing D-chiro-inositol or a derivative or metabolite thereof. Preferably, the inventive method comprises administering to a mammal an effective amount of D-chiro-inositol.

A third preferred embodiment of the present invention is directed to compositions and methods for decreasing the risk of or preventing impairment of intellect due to conditions associated with neural tube disorders, including hypoplasia or aplasia of cranial nerve nuclei, obstruction of the flow of cerebrospinal fluid flow within the ventricular system, cerebellar dysplasia, disordered migration of cortical neurons, fusion of the thalami, genesis of the corpus callosum, and complete or partial agenesis of the olfactory tract and bulb, whether or not caused by diabetes or hyperglycemia.

In this embodiment, the inventive composition comprises an effective amount of D-chiro-inositol, or a suitable derivative or metabolite thereof, or a compound containing D-chiro-inositol or a derivative or metabolite thereof, and an acceptable carrier. Preferably, the inventive composition comprises D-chiro-inositol.

The inventive method comprises administering to a mammal in need thereof an effective amount of D-chiro-inositol, or a suitable derivative or metabolite thereof, or a compound containing D-chiro-inositol or a derivative or metabolite thereof. Preferably, the inventive method comprises administering to a mammal an effective amount of D-chiro-inositol.

Other preferred embodiments of the present invention are directed to compositions and methods for decreasing the risk of or preventing abnormal neurulation characterized by neural tube disorders, enhancing posterior neuropore closure, decreasing the risk of or preventing failure of posterior neuropore closure, decreasing the ratio of posterior neuropore length to crown-rump length, decreasing the risk of or preventing failure of the neural tube to close at the rostral end in mammals which results in NTDs characterized by spina bifida, and decreasing the risk of or preventing failure of the neural tube to close at the caudal end in mammals which results in NTDs characterized by anencephaly.

In these embodiments, the inventive compositions comprise an effective amount of D-chiro-inositol, or a suitable derivative or metabolite thereof, or a compound containing D-chiro-inositol or a derivative or metabolite thereof, and an acceptable carrier. Preferably, the inventive compositions comprise D-chiro-inositol.

The inventive methods comprise administering to a mammal in need thereof an effective amount of D-chiro-inositol, or a suitable derivative or metabolite thereof, or a compound containing D-chiro-inositol or a derivative or metabolite thereof. Preferably, the inventive methods comprise administering to a mammal an effective amount of D-chiro-inositol.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference.

### Comparative Studies of the Effects of D-Chiro-Inositol in Comparison with Control and Myo-Inositol Treated Curly-Tail Mouse Embryos in vivo and in vitro were Performed

In in vitro studies, curly-tail mouse embryos were explanted into culture at 9.5 days after conception, corresponding to the onset of low spinal cord neural tube closure. Inositol additions were made at the start of culture and were maintained throughout the 24 hour culture period. Inositols were maintained at 20 μg/ml. At the end of culture, the embryos were dissected from their extraembryonic membranes, and crown-rump and posterior neuropore lengths were measured as indices of developmental growth, progression and low spinal neural tube closure.

In in vivo studies, pregnant curly-tail female mice were treated with inositols or PBS (control) by either subcutaneous injection or oral gavage. Using inositols at 75 mg/ml injected by subcutaneous minipump at μL/hour, a total daily dosage of 90 mg/kg/day was administered. Inositols were administered from gestational day 8.5 through day 18.5, and fetuses were scored for NTDs on days 12.5 and 18.5. For oral dosing, mice were administered via gavage with 400 mg/kg of inositol twice daily on gestational days 8.5 through 10.5, with fetuses scored for NTDs on day 12.5.

Results of the studies are summarized below:

1 Mean posterior neuropore to crown-rump length ratio in embryonic curly tail mice following 24 hours of treatment with D-chiro-inositol at 20 μg/ml (.029±.018) decreased 5.6-fold compared to control (.168±.03.8) or myo-inositol treated (0.172 ±0.026) mice, and mean posterior neuropore length decreased 6-fold (0.591±0.130 for control, 0.601±0.090 for myo-inositol treated, 0.095±0.055 for D-chiro-inositol treated) following 24 hours of treatment with D-chiro-inositol in vitro.

2. Four to ten days of treatment with D-chiro-inositol by subcutaneous administration of 90 mg/kg/day reduced the number of curly tail mouse fetuses showing NTDs characterized by spina bifida from 9 in control (I 5.3%) and 4 in myo-inositol treated mice (6.3%) to 1 (1.7% of D-chiro-inositol treated mice), 9-fold and 4-fold decreases (p>0.05), respectively. Three days of treatment with D-chiro-inositol by oral administration reduced the number of curly tail mouse fetuses showed NTDs characterized by spina bifida from 8 in control (18.2%) and 3 in myo-inositol treated mice (8.6%) to 1 (2.3% of D-chiro-inositol treated group), 8-fold and 3-fold decreases (p>0.05), respectively.

### Comparison of Administration Route of D-Chiro-Inositol in Treating Folate Acid Resistant Neural Tube Defects

Mice—Curly tail mice are maintained as a homozygous, random-bred stock (Van Straaten, H. W. M. & Copp, A. J., Anat. Embryol. 203, 225-237 (2001)) Experimental litters were generated by timed matings, and the day of finding a copulation plug was designated embryonic day (E) 0.5.

In vitro inositol treatment—E9.5 embryos (17-19 somite stage) were cultured for 24 hours at 38° C. in whole rat serum. Thirty minutes after the start of culture, myo-inositol (Sigma, UK) or D-chiro-inositol (Insmed, Va., USA) was added to the medium (62.5 μl of inositol stock per ml rat serum) to a final concentration of 5, 10, 20 or 50 μg/ml inositol. Control cultures received an equal volume of phosphate buffered saline (PBS). Following culture, embryos were scored for: (i) posterior neuropore length (the distance from the rostral end of the posterior neuropore to the tip of the tail bud), (ii) crown-rump length and (iii) somite number.

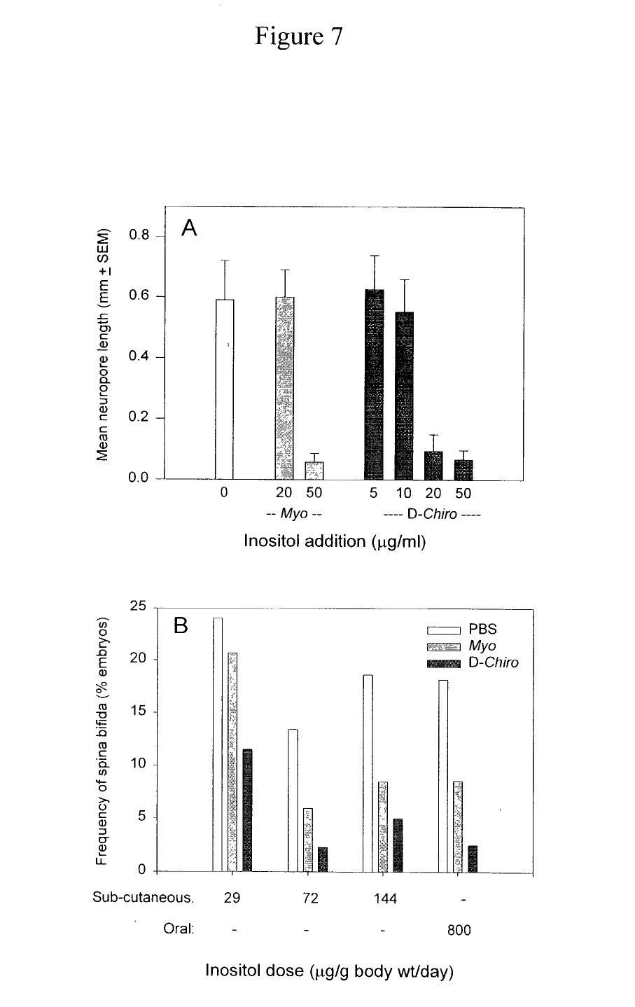

E9.5 embryos represent the period during which the neural tube is closing at the posterior neuropore of the mouse embryo. In curly tail embryos, neuropore closure is delayed or fails to be completed, leading to the development of tail flexion defects and spina bifida, respectively. Both myo- and D-chiro-inositol exhibited a dose-dependent normalisation of posterior neuropore length in embryo culture, as judged by the reduction in neuropore length observed in embryos treated with higher inositol doses. Strikingly, D-chiro-inositol reduced neuropore length at both 20 and 50 μg/ml whereas a comparable effect was seen with myo-inositol only at 50 μg/ml. Embryos exposed to 20 μg/ml myo-inositol (and 5-10 μg/ml D-chiro-inositol) exhibited neuropore lengths that were not different from PBS-treated controls (FIG. 7A). Moreover, we found no significant difference in crown-rump length of embryos treated with either myo- or D-chiro-inositol, compared with PBS-treated controls, suggesting that the effect of inositol is specific to the closing posterior neuropore, and not mediated via alteration of embryonic growth.

In utero inositol treatment (subcutaneous administration)—For sub-cutaneous administration, osmotic mini-pumps (capacity 100 μl, delivery rate 1 μl/hour; Model 1003D, Alzet) were filled with solutions of 30, 75 or 150 mg/ml inositol (delivering 29, 72 and 144 μg inositol/g body weight/day respectively for a 25 g mouse), or PBS as a control. Mini-pumps were incubated in sterile PBS at 37° C. for 4 hours and then implanted subcutaneously on the back of pregnant mice at E8.5. General anesthesia was induced by an intra-peritoneal injection of 0.01 ml/g body weight of a solution comprising 10% Hypnovel® (midazolam 5 mg/ml) and 25% Hypnorm® (fentanyl citrate 0.315 mg/ml, fluanisone 10 mg/ml) in sterile distilled water.

Pregnant females were killed at E18.5, and the total number of implantations, classified as viable fetuses or resorptions, was recorded. Fetuses were dissected from the uterus and inspected immediately for the presence of open lumbo-sacral spina bifida and tail flexion defects: the primary manifestations of the curly tail genetic defect.

Using surgically implanted osmotic mini-pumps to administer inositol at a constant rate over a period of 72 hours of pregnancy, encompassing the stages of neural tube closure, we observed a dose-dependent effect of both myo- and D-chiro-inositol. At 29 μg/g body weight/day, neither myo- nor D-chiro-inositol significantly affected the frequency of fetuses with spina bifida (FIG. 7B), whereas at dosing levels of 72 and 144 μg/g body weight/day both inositols reduced the frequency of spina bifida compared with PBS-treated pregnancies (FIG. 7B). At these higher dose levels, inositol caused a significant shift towards the mild end of the NTD phenotype spectrum (Table 1). As with the in vitro study, D-chiro-inositol appeared most effective, causing a 73-83% decrease in spina bifida frequency relative to PBS controls, at both 72 and 144 μg/g body weight/day. Myo-inositol produced a consistent 54-56% reduction in spina bifida frequency (FIG. 7B).

TABLE 1
Frequency of neural tube and tail defects among curly tail
fetuses treated in utero with myo- and D-chiro-inositol

| | | | Phenotype of fetuses^{2,3} | | |
|---|---|---|---|---|---|
| | | | Spina | | |
| Route of | Treat- | Inositol | bifida ± | | |
| administration | ment | dose¹ | curly tail | Curly tail | Normal |
| Sub- | PBS | — | 6 (24.0) | 14 (56.0) | 5 (20.0) |
| cutaneous | myo | 29 | 6 (20.7) | 12 (41.4) | 11 (37.9) |
| | D-chiro | 29 | 3 (11.5) | 17 (65.4) | 6 (23.1) |
| | PBS | — | 14 (13.5) | 63 (60.6) | 27 (25.9) |
| | myo | 72 | 4 (6.0) | 37 (55.2) | 26 (38.8) |
| | D-chiro | 72 | 2 (2.3) | 39 (45.4) | 45 (52.3) |
| | PBS | — | 19 (18.6) | 49 (48.0) | 34 (33.3) |
| | myo | 144 | 8 (8.5) | 52 (55.3) | 34 (36.2) |
| | D-chiro | 144 | 5 (5.0) | 46 (46.5) | 48 (48.5) |
| Oral | PBS | — | 8 (18.2) | 21 (47.7) | 15 (34.1) |
| | myo | 800 | 3 (8.6) | 15 (42.9) | 17 (48.6) |
| | D-chiro | 800 | 1 (2.6) | 12 (30.8) | 26 (66.7) |

¹Inositol dose is expressed as: μg inositol/g body weight/day.
²Phenotype frequencies expressed as: number of fetuses (% of total for treatment group).
³Statistical analysis: distribution of embryos among the three phenotype categories varies significantly with treatment group at 72, 144 and 800 μg inositol/g body weight/day (Chi-square tests, p = 0.0005, 0.0051 and 0.026 respectively) but not at 29 μg inositol/g body weight/day (p = 0.34).

In utero inositol treatment (oral dosing)—For oral administration, pregnant mice were gavaged with 0.5 ml inositol solution in PBS at 12 hourly intervals from E8.5 to E10.5 (six doses in total; 800 μg/g body weight/day).

Pregnant females were killed at E18.5, and the total number of implantations, classified as viable fetuses or resorptions, was recorded. Fetuses were dissected from the uterus and inspected immediately for the presence of open lumbo-sacral spina bifida and tail flexion defects: the primary manifestations of the curly tail genetic defect.

Inositol was administered orally to pregnant females, using a twice-daily dosing regime, from E8.5 to E10.5, that delivered 800 μg/g body weight/day. As with sub-cutaneous administration, we detected a marked reduction in the frequency of spina bifida among the offspring of mice treated with either myo- or D-chiro-inositol (FIG. 7B), and a significant shift in the distribution of fetuses between the three phenotype categories (Table 1). D-chiro-inositol was most effective, causing a 86% reduction in the frequency of spina bifida, while a 53% reduction was observed for myo-inositol.

We investigated whether clustering of fetuses of particular phenotypes within litters may have affected the outcome of the comparison between myo-inositol, D-chiro-inositol and PBS. An ordinal multilevel regression model (which took into account the potential non-independence of fetuses within litters) confirmed that fetuses treated subcutaneously with D-chiro-inositol are significantly more likely to be normal than those treated with PBS (p<0.0005). The comparison between myo-inositol and PBS also reached statistical significance in this analysis (p<0.002). In the oral dosing study, fetuses were more likely to be normal when treated with D-chiro-inositol (p=0.0097) than PBS, whereas the values for myo-inositol and PBS did not differ significantly (p=0.13). Importantly, the multilevel analysis showed that the difference between treatment groups is unaffected when possible litter effects are taken into account.

One possible explanation for a decrease in spina bifida frequency following maternal inositol administration could be an increase in loss of affected fetuses during pregnancy. We examined both resorption rate and litter size in pregnancies receiving either subcutaneous or oral inositol, and found no significant difference between pregnancies treated with inositol and those receiving PBS alone (Table 2).

TABLE 2
Survival of embryos and fetuses among curly tail litters treated
in utero with myo- and D-chiro-inositol

| Route of | | Inos- | | No. | | Mean litter |
|---|---|---|---|---|---|---|
| admin- | Treat- | itol | No. | viable | No. uterine | size ± |
| istration | ment | dose¹ | litters | fetuses | resorptions² | SEM³ |
| Sub- | PBS | — | 4 | 25 | 3 (10.7) | 6.25 ± 1.03 |
| cutaneous | myo | 29 | 4 | 29 | 2 (6.5) | 7.25 ± 1.80 |
| | D-chiro | 29 | 3 | 26 | 1 (3.7) | 8.67 ± 1.76 |
| | PBS | — | 12 | 104 | 10 (8.8) | 8.67 ± 0.50 |
| | myo | 72 | 8 | 67 | 7 (9.5) | 8.38 ± 0.78 |
| | D-chiro | 72 | 12 | 86 | 8 (8.5) | 7.17 ± 0.89 |
| | PBS | — | 12 | 102 | 9 (8.1) | 8.50 ± 0.79 |
| | myo | 144 | 12 | 94 | 9 (8.7) | 7.83 ± 0.44 |
| | D-chiro | 144 | 14 | 99 | 6 (5.7) | 7.07 ± 0.75 |
| Oral | PBS | — | 7 | 44 | 5 (10.2) | 6.29 ± 0.36 |
| | myo | 800 | 6 | 35 | 3 (7.9) | 5.83 ± 0.48 |
| | D-chiro | 800 | 6 | 39 | 3 (7.1) | 6.50 ± 1.09 |

¹Inositol dose is expressed as: μg inositol/g body weight/day.
²Values in parentheses: resorptions as % of total number of implantations (i.e. viable fetuses + resorptions). Logistic regression analysis: the proportion of resorptions does not differ significantly between myo-inositol and PBS litters (p = 0.87), between D-chiro-inositol and PBS litters (p = 0.33), or between inositol dose levels (p = 0.88).
³Litter size (viable fetuses per litter) does not vary significantly with treatment group or inositol dose level (two-way analysis of variance: treatments, p = 0.987; inositol dose level p = 0.054). Litters treated with oral inositol contain an average of 1.6 (95% confidence intervals: 0.4, 2.8) fewer animals than litters treated with sub-cutaneous inositol.

Discussion

In this study, we have evaluated the ability of exogenous inositol to prevent spinal NTD in the folate-resistant curly tail mouse genetic model. Maternal inositol administration significantly reduces the frequency of spina bifida in curly tail mice and normalises closure of the posterior neuropore in whole cultured embryos. A striking finding is the increased potency of D-chiro- compared with myo-inositol, two closely related enantiomers that differ only in the orientation of the carbon-two hydroxyl group (C2-OH) relative to the plane of the six carbon ring. At identical dosage levels, subcutaneously administered D-chiro-inositol causes a consistently greater reduction in frequency of spina bifida than myo-inositol. Moreover, in vitro, D-chiro-inositol is effective in normalising neural tube closure at a concentration at which myo-inositol has no effect. The greater preventive effect of D-chiro-inositol may result from its differential incorporation and metabolism within the phosphoinositide cycle. Insulin stimulation of rat fibroblasts expressing the human insulin receptor leads to a significant increase in the incorporation of D-chiro-inositol into phospholipids whereas the effect on myo-inositol incorporation is only marginal (Pak, Y., Paule, C. R., Bao, Y. D., Huang, L. C. & Larner, J., Proc. Natl Acad. Sci. USA 90, 7759-7763 (1993)). Moreover, D-chiro-inositol induces a much larger reduction in plasma glucose level in rats rendered diabetic by streptozotocin administration compared with exogenous myo-inositol (Ortmeyer, H. K., Huang, L. C., Zhang, L., Hansen, B. C. & Larner, J., Endocrinology 132, 646-651 (1993)). In humans, D-chiro-inositol can increase the action of insulin in patients with polycystic ovary syndrome, improving ovulatory function, reducing blood pressure, and decreasing blood androgen and triglyceride concentrations (Nestler, J. E., Jakubowicz, D. J., Reamer, P., Gunn, R. D. & Allan, G. (1999) N. Engl. J. Med. 340, 1314-1320). These findings suggest an inherently greater potency or bioactivity of D-chiro-inositol than myo-inositol, perhaps as a result of incorporation into different phosphatidylinositol species. It is striking, however, that these differences of in vivo potency are maintained in the face of the demonstrated interconversion of the two inositol isomers (Pak, Y., Huang, L. C., Lilley, K. J. & Larner, J. (1992) J. Biol. Chem. 267, 16904-16910). Perhaps the rate of interconversion is too low to obscure the inherently greater potency of D-chiro-inositol in short-term effects on embryonic development.

What is claimed is:

1. A method for decreasing the risk of or preventing neural tube disorders in a mammal comprising administering to a mammal an effective amount of D-chiro-inositol or a derivative or metabolite thereof.

2. The method of claim 1, wherein D-chiro-inositol is administered.

3. The method of claim 1, wherein said effective amount of D-chiro-inositol is in the range of about 3 to about 300 mg/kg/day.

4. The method of claim 3, wherein said effective amount of D-chiro-inositol is in the range of about 5 to about 180 mg/kg/day.

5. The method of claim 1, wherein an effective amount of folic acid is administered with said D-chiro-inositol.

6. The method of claim 5 wherein the effective amount of folic acid is about 0.5 mg/day to about 5.0 mg/day.

7. The method of claim 6, wherein the effective amount of folic acid is about 1.0 mg/day to about 4.0 mg/day.

8. A method for decreasing the risk of or preventing neural tube disorders in a mammal comprising administering to a mammal a composition comprising an effective amount of D-chiro-inositol, or a derivative or metabolite thereof, and an effective amount of folic acid.

9. A composition comprising:
(a) about 25 to about 1000 milligrams of a compound selected from the group consisting of D-chiro-inositol; D-chiro-inositol phosphate; D-chiro-inositol ester; D-chiro-inositol ether; D-chiro-inositol acetals; D-chiro-inositol ketals; fagopyritol; and D-chiro-inositol amino dissacharides: and
(b) about 1 to about 5 milligrams of folic acid.

10. The composition of claim 9 wherein the compound is D-chiro-inositol.
